(19) United States
(12) Patent Application Publication
(10) Pub. No.: US 2007/0173939 A1
(43) Pub. Date: Jul. 26, 2007

(54) SYSTEMS AND METHODS FOR FIXATION OF BONE WITH AN EXPANDABLE DEVICE

(75) Inventors: Daniel H. Kim, Los Altos, CA (US); J. Christopher Flaherty, Topsfield, MA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/609,223

(22) Filed: Dec. 11, 2006

Related U.S. Application Data
(60) Provisional application No. 60/753,711, filed on Dec. 23, 2005.

(51) Int. Cl. A61F 2/44; A61F 2/28

(52) U.S. Cl. 623/17.11; 623/23.54

(57) ABSTRACT

Apparatus, systems and methods for providing fixation to an interior body region of a patient are provided. The apparatus includes an expandable cage which is inserted into a body region, such as cancellous bone of a vertebra, in a first geometry, and transitioned to a second geometry. Delivery devices and other tools are included including a material delivery device used to inject an agent into a body region without requiring or creating a void in that region. Methods are also provided for providing fixation to an internal body region.

### CROSS-REFERENCES

This application claims the benefit of U.S. Provisional Application No. 60/753,711, filed Dec. 23, 2005, which application is incorporated herein by reference.

### FIELD OF THE INVENTION

The present invention generally relates to surgical instruments and methods for using these instruments. More particularly, but not exclusively, minimally invasive apparatus and methods of fixation of one or more bone structures is disclosed.

### BACKGROUND OF THE INVENTION

Systems, methods and devices for fixation of bone have been available for many years. Approximately 700,000 vertebral fractures associated with osteoporosis occur each year. Traditional conservative management of vertebral body compression fractures includes analgesics, immobilization, muscle relaxants, physical therapy, and external bracing maybe helpful but often significant number of patients still remain symptomatic. Current percutaneous vertebroplasty is radiologically guided, therapeutic procedure for the treatment of pain by injecting methyacrylate cement into fractured vertebral body. Current method of vertebroplasty does not restore the height loss in a compressed segment. Often fractures in multiple levels can result in progressive anterior column shortening and thoracolumbar angulation. This can potentiate disability, lung dysgunction, and eating disorders with development of deformity. There is kyphoplasty method using an inflatable balloon tamp inserted into the vertebral body and distract the fracture vertebral body to create a void then fill with methyacrylate cement. This procedure may restore some height loss but often not adequate enough to support the biomechanical load.

This present invention provides a surgical instruments and methods for using invented instruments to provide internal stabilization of the fractured spine to restore the alignment with insertion of bone cements by minimally invasive procedure techniques.

There remains a need for minimally invasive methods and apparatus for bone fixation procedures, including but not limited to vertebral segment fixation procedures. There is a need for procedures that are simple to perform and reliably achieve the desired safe and effective outcome. Goals of these new procedures and instruments include minimally invasive percutaneous techniques in order to shorten recovery times, improve procedure success rates and reduce the number of resultant adverse side effects.

### SUMMARY OF THE INVENTION

According to a first aspect of the invention, an apparatus for providing fixation of an interior body region of a patient is disclosed. The apparatus comprises an expandable cage comprising a proximal ring, a distal ring, and a plurality of elongate members. Each elongate member includes a proximal end, a distal end, and a middle portion therebetween. Each of the proximal ends are attached to the proximal ring, and each of the distal ends are attached to the distal ring of the expandable cage. The plurality of elongate members are transitionable from a first geometry in which the middle portions define a first, compact geometry, to a second geometry in which the middle portions define a second, expanded geometry. The area defined by the middle portion of the elongate members approximates a smaller area in the first geometry than the area approximated in the second geometry. The areas approximated in the first geometry may include a circle, a rectangle or an oval.

In a preferred embodiment, the expandable cage is configured to provide structural reinforcement to the interior body region in which it is placed. The expandable cage transitions from the first geometry to the second geometry either by creating a space or void within the region, or preferably by cutting through bone or other tissue without creating a void. The apparatus may further include reinforcing material inserted within the internal space surrounded by the elongate members, prior to, during, or after the elongate members transition from the first geometry to the second geometry. Numerous biocompatible materials may be used, singly or in combination, to construct the expandable cage, such as metals and non-metals. The elongate members may include sharpened edges or other enhancement to assist in cutting through bone, such as the cancellous bone of a vertebral segment. The expandable cage preferably includes a lumen allowing insertion over a guidewire such as a K-wire inserted into a vertebra of the spine of the patient.

The expandable cage includes an attachment element for attaching to a delivery device, a pushing or pulling device, a reinforcing material dispensing device and/or another percutaneously inserted device. The attachment element may be integral to the proximal ring, the distal ring, or a location in between, and may include threads, a fusible link, a frictionally engaging hole, a bayonet lock and/or other engagement means allowing engagement and/or disengagement of the device with the expandable cage, and preferably re-engagement such as re-engagement at a subsequent step of the fixation procedure. The expandable cage can be transitioned from the first geometry to the second geometry by applying a pushing force to the proximal ring, a pulling force to the distal ring, or a combination of the two forces. Alternatively, an expanding element, such as an inflatable balloon or an expandable cage, can be introduced within the space surrounded by the elongate members and expanded to apply a radial outward force to the elongate members. Alternatively or additionally, injection of expanding reinforcing material can be made into the internal space, the expansion of the material causing the elongate members to transition from the first geometry to the second geometry. In a preferred embodiment, the expandable cage is delivered into the interior body region along the same axis as a delivery device attached to the expandable cage. In another preferred embodiment, the elongate members radially expand to the second geometry, the radial expansion orthogonal to the axis of the delivery device.

The elongate members may comprise various cross sections such as rectangular, oval, triangular or circular cross sections. The cross section of a first elongate member may differ from the cross section of a second elongate member, such as a change in width, thickness, diameter, major axis length, minor axis length or a change in material of construction.

In another preferred embodiment, the expandable cage may be removed, such as at the end of the procedure, or in a subsequent procedure. In another preferred embodiment, the expandable cage may include a covering, such as a covering that remains in place if the expandable cage is removed. Agents such as bone reinforcing material agents may be introduced within the implanted mesh. The mesh may be constructed of one or more biocompatible materials including but not limited to: metal such as superelastic metal such as superelastic Nitinol; woven polyester such as Dacron; graphite and other carbon fibers; para-aramid fibers such as Kevlar; fluorocarbon fibers; polyethylene fibers such as ultra high molecular weight polyethylene fibers such as Dyneema; heat fused fibers; synthetic fibers; and combinations thereof.

In yet another preferred embodiment, the expandable cage is resiliently biased in the first geometry, the second geometry, or a geometry in between the first and second geometries.

In yet another preferred embodiment, the proximal ring, the elongate members, and the distal ring, have similar construction properties. These construction properties include but are not limited to: thickness; diameter; major axis length; minor axis length; material of construction; flexibility; and combinations thereof.

In yet another preferred embodiment, the distal ring includes an enhancement of its distal end to assist in advancement through bone, such as advancement made while the elongate members are in the first, compact geometry. Enhancements include sharpening, adding of a serrated edge, and delivery of electrical energy such as monopolar or bipolar energy. The enhancements are preferably configured to assist in advancement through cancellous bone but not cortical bone, such as the cancellous and cortical bone portions of a vertebra of the spine.

In another aspect of the present invention, a method of manufacturing an expandable cage for placement into an interior body region of a patient is disclosed. The method comprises creating a hollow tube with a proximal end and a distal end. Slots are created through the side of the tube, from a point distal to the proximal end to a point proximal to the distal end. The material between the slots functions as elongate members which are expandable from a first geometry defining a first area and a second geometry defining a second area. The second area is greater than the first area. The area in the first geometry and/or the second geometry may approximate a circle, an oval, a rectangle or other shape. The creation of the tube can be accomplished with an extrusion process, rolling and welding of a sheet or machining, drilling or etching of a solid cylinder. The slots can be accomplished with a machining, laser cutting or etching process. The method of manufacture may further comprise processing the expandable cage to cause a resiliently biased condition, such as the first geometry, the second geometry, or a geometry in between. The method of manufacture may further comprise additional processing of the elongate members, such as a sharpening or other process to assist in the elongate members cutting through bone or other tissue when transitioning from the second geometry to the first geometry. The method of manufacture may further comprise creating an attachment element on the proximal ring, the distal ring or a location in between, such that a device such as a delivery device can be removably attached to the expandable cage. The method of manufacture may further comprise processing the expandable cage to change one or more properties of the expandable cage, such as an annealing process used to make the expandable cage more rigid.

In another aspect of the present invention, a method of providing fixation to an interior body region of a patient is disclosed. A fixation apparatus of the present invention is inserted into the interior body region of a patient, such as within a bone of a limb of the patient, or within a vertebra of the spine of the patient. The fixation apparatus may be inserted in a fractured bone segment, or an impending fracture bone segment. The fixation apparatus may be inserted into an osteoporotic bone segment. The elongate members of the expandable cage of the apparatus are transitioned from the first geometry to the second geometry. The method may further comprise the step of placing reinforcing material in the space surrounded by the elongate members. The reinforcing material can take on or more numerous forms: including bone penetrating agents, bone material including bone biologic material; material that hardens over time; compounds including two mixed parts such as two-part epoxy or cement, or other agents. The expandable cage may include a covering, such as a metal covering configured to cut through bone. The expandable cage may be removed leaving the covering implanted.

In another aspect of the present invention, a device for placing material into an interior body region of a patient is disclosed. The device comprises an elongate tube, a body material pulverizing assembly, a vacuum assembly and an agent delivery assembly. The elongate tube has a proximal end and a distal end. The body pulverizing assembly is deployable from the distal end of the elongate tube and is configured to contact and pulverize body material proximate the distal end of the elongate tube. The vacuum assembly is configured to remove the pulverized body material from the internal body region. The agent delivery assembly is configured to deliver an agent, such as a reinforcing agent, to the interior body region, in the space previously occupied by the pulverized material. The agent is placed in the interior body region prior to a space, a void, having been created. The body pulverizing assembly may include an auger assembly that can be advanced and retracted by an operator, and including cutting threads which direct the pulverized material into a lumen of the elongate tube. Each of the body material pulverizing assembly, the vacuum assembly, and the agent delivery assembly can be activated using one or more controls, such as a single control which activates each of the assemblies simultaneously. In an alternative embodiment, the agent delivery assembly can be activated independently, in other words it can be activated without the body material pulverizing assembly being on. In preferred embodiments, the agent delivery assembly must be on when either the body material pulverizing assembly or the vacuum assembly are activated, avoiding the creation of any void space prior to agent injection.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 illustrates a perspective view of an expandable cage shown in an unexpanded condition, consistent with the present invention.

FIG. 2 illustrates a perspective view of the expandable cage of FIG. 1, shown in an expanded condition, consistent with the present invention.

FIG. 3 illustrates a perspective view of an expandable cage with a biocompatible covering, shown in an unexpanded condition, consistent with the present invention.

FIG. 4 illustrates a perspective view of the expandable cage of FIG. 3, shown in an expanded condition, consistent with the present invention.

FIG. 5 illustrates a side view of a spine portion of a healthy patient including three vertebral segments.

FIG. 6 illustrates a side view of a spine portion of a patient with a fractured middle vertebral segment.

FIG. 7 illustrates a side view of an initial step of a fixation procedure treating the fractured vertebral segment of FIG. 6 wherein an unexpanded expandable cage connected to the distal end of a delivery tube is introduced into cancellous bone, consistent with the present invention.

FIG. 8 illustrates a side view of a subsequent step of a fixation procedure wherein the expandable cage has been partially expanded, with elongate members cutting through the cancellous bone, consistent with the present invention.

FIG. 9 illustrates a side view of a subsequent step of a fixation procedure wherein the expandable cage has been fully expanded, consistent with the present invention.

FIG. 10 illustrates a magnified view of the expandable cage of FIG. 9.

FIG. 11 illustrates a side view of a subsequent step of a fixation procedure including an alternative expandable cage design including a mesh covering, wherein a material delivery tool has been advanced to the expandable cage over a guidewire and reinforcing material is being introduced within the expandable cage, consistent with the present invention.

FIG. 12 illustrates a side view of a subsequent step of a fixation procedure wherein the material delivery tool has been removed after having deposited sufficient reinforcing material within the expandable cage, consistent with the present invention.

FIGS. 13 through 16 illustrate side views of a series of steps of a fixation procedure similar to the steps illustrated in FIGS. 9 through 12 wherein the expandable cage is removed, as shown in FIG. 14, prior to introducing the reinforcing material into a covering previously surrounding the expandable cage, consistent with the present invention.

FIG. 17 illustrates a side view of an expandable cage with a sharpened distal edge, consistent with the present invention.

FIG. 18 illustrates a side view of an expandable cage with proximal and distal plugs, consistent with the present invention.

FIG. 19 illustrates a side view of a material dispenser device with an auger assembly and a simultaneous material delivery assembly, consistent with the present invention.

FIG. 20 illustrates an expandable cage delivery device including an integral forward cutting element, consistent with the present invention.

### DETAILED DESCRIPTION OF THE INVENTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Reference will now be made in detail to the present embodiments of the invention, examples of which are illustrated in the accompanying drawings. Wherever possible, the same reference numbers will be used throughout the drawings to refer to the same or like parts.

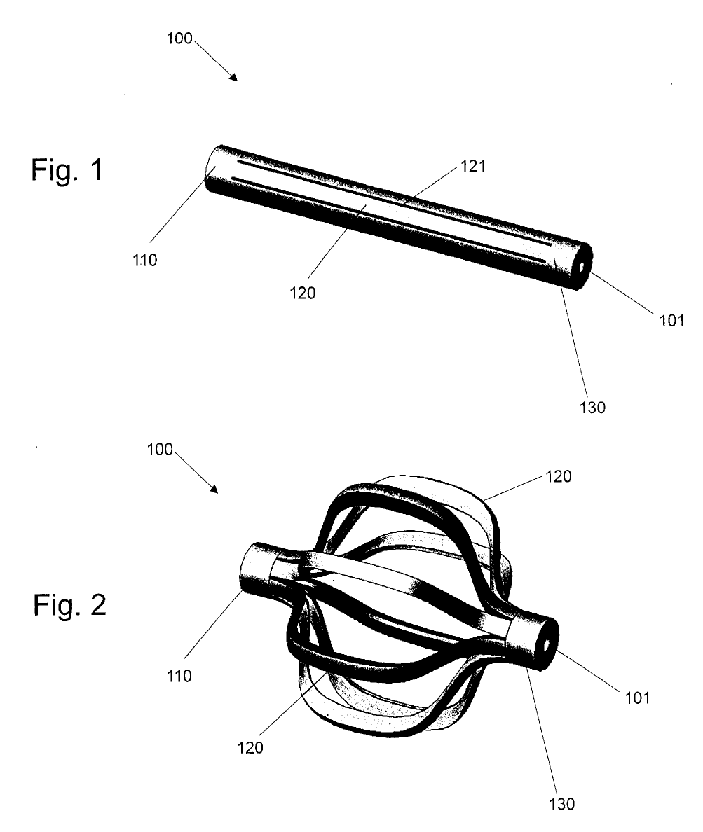

The present invention will now be described in greater detail by way of the following description of exemplary embodiments and variations of the systems and methods of the present invention. Referring now to FIG. 1, expandable cage 100 is illustrated in a first geometry configured for delivery into an interior body region of a patient to subsequently provide fixation to that body region. Expandable cage 100 is preferably a metal tube constructed of materials such as Nitinol or stainless steel. Expandable cage 100 includes multiple slots, 121, such as slots made by a laser cutting process performed on an extruded Nitinol tube. Forming of the slots 121 creates multiple elongate members 120 having a width defined by the separation between the associated slots 121. In a preferred embodiment, slots 121 are separated by equal distances creating elongate members 120 with similar widths. In an alternative embodiment, slots 121 are separated by varied distances creating elongate members 120 with varied widths.

On each end of expandable cage 100 are continuous rings, proximal ring 110 and distal ring 130. A guidewire lumen 101 passes through expandable cage 100 entering proximal ring 110 and exiting distal ring 130. Proximal ring 110 includes an attachment element, not shown but described in detail in reference to subsequent figures. This attachment element is preferably a threaded hole which rotationally attaches to a push rod used to introduce expandable cage 100 into an interior body region of a patient. In an alternative embodiment, the push rod is attached with a fusible link such as a fusible link which is detached by applying an electrical current. In another alternative embodiment, the push rod is frictionally engaged with the attachment element of proximal ring 110, such that sufficient retraction force causes the push rod to disengage from expandable cage 100. Additionally or alternatively, distal ring 130 may include an attachment element, for attachment to a push rod used to introduce expandable cage 100 into an interior body of a patient. The push rod may pass through an opening in the proximal ring, through a lumen defined by elongate members 120, and attach to an element of distal ring 130. Similar to the attachment element described in reference to proximal ring 110, the attachment element of distal ring 130, also not shown, can include various engagement mechanisms such as rotational elements including a threaded hole, a fusible link, a bayonet lock, frictional engagement portions, and combinations thereof.

In a preferred embodiment, proximal ring 110, elongate members 120 and distal ring 130 are all constructed of a single material, such as Nitinol. In an alternative embodiment, proximal ring 110, elongate members 120 and/or distal ring 130 include different materials of construction, such as materials selected from the group consisting of: metals such as stainless steel and Nitinol; composites such as graphite composites; plastics such as metal reinforced plastics and combinations thereof. Other construction properties may be similar or dissimilar between proximal ring 110, elongate members 120 and distal ring 130 such as parameters selected from the group consisting of: thickness; width; diameter; major axis length; minor axis length; flexibility; and combinations thereof. In a preferred embodiment, distal ring 130 includes a sharpened distal end, not shown, such as an end sharpened to improve delivery of expandable cage 100 into bone. Expandable cage 100 is configured to be delivered percutaneously, typically with a diameter of 4 mm or less in the first geometry configuration shown in FIG. 1. In a preferred embodiment, the diameter of expandable cage 100 in the first geometry of FIG. 1 is between 3 and 8 mm. In an alternative embodiment, this diameter is between 1 and 10 mm. Expandable cage 100 includes lumen 101, configured to allow expandable cage 100 to pass over a guidewire such as a guidewire used to advance devices into a vertebral segment of the spine.

Expandable cage 100 may be biased, including resiliently biased conditions, to be in the geometric configuration of FIG. 1, or may have forces applied, such as radially constraining forces applied by a surround tube, to place expandable cage 100 in the geometry shown in FIG. 1. Expandable cage 100 is preferably manufactured from a tube, such as an extruded tube; a tube constructed by rolling a flat metal sheet and welding the contacting seam; or a tube that is machined, drilled such as laser drilled, and/or etched from a metal rod. Slots 121 can also be manufactured utilizing various processes including machining; sawing; laser cutting; etching; and combinations thereof. Expandable cage 100 may have a circular cross section as shown, or other cross sectional shapes such as ovals and rectangles. In an alternative embodiment, the external surface of expandable cage is modified, such as a modification selected from the group consisting of: removal of material such as etching or machining; plating; modification of surface energy; modification of surface texture; and combinations thereof. In another alternative embodiment, material is added to the elongate members 120, such as material added utilizing anodizing; plasma deposition; ion beam assisted deposition; sputtering; and combinations thereof. In another alternative embodiment, the expandable cage 100 is treated to modify its stiffness, such as an annealing process performed to increase rigidity of the elongate members 120. In another alternative embodiment, the elongate members 120 are modified in a process that sharpens the elongate members 120 to better cut through bone such as cancellous bone of a vertebral segment. In another alternative embodiment, the elongate members 120 include means of attaching to a source of electrical power, attachment means not shown, such that the elongate members 120 can be electrified to better cut through bone when the mid portions of elongate members 120 expand as described in detail herebelow. In this alternative embodiment, energy such as monopolar and/or bipolar radiofrequency energy can be applied to the elongate members 120 to cut through bone.

Referring now to FIG. 2, expandable cage 100 is illustrated in a second geometry configured to provide fixation to the interior body region in which it was inserted in the first geometry of FIG. 1. The second geometry of FIG. 2 includes the mid portions of elongate members 120 defining a diameter that is greater than the diameter defined by the first geometry of the elongate members 120 shown in FIG. 1. In a preferred embodiment, expandable cage 100 has been modified from the first geometry of FIG. 1 to the second geometry of FIG. 2 by providing an axial force to proximal ring 110 and/or distal ring 130, such as by an attached rod not shown but described in detail in reference to subsequent figures herebelow. Expandable cage 100 is preferably made of a metal alloy, such as the nickel titanium alloy Nitinol. Expandable cage 100 may be resiliently biased in the first geometry of FIG. 1, the second geometry of FIG. 2, or in a geometry somewhere between the first geometry and the second geometry. In embodiments in which the elongate members 120 have a mid portion defining a diameter greater than the associated diameter of FIG. 1, elongate members 120 may be constrained such as within a tube for percutaneous introduction into a fractured bone segment of a patient, such as a femur or vertebral segment. Expandable cage 100 is preferably resiliently biased using a heat treating process, such as a heat treating process common to treatment of Nitinol when used in superelastic applications. Proximal ring 110 and/or distal ring 130 may additionally or alternatively be processed to enhance superelastic properties or creating a resiliently biased geometry.

Elongate members 120 are configured to provide structural reinforcement to the interior body region in which expandable cage 100 is placed, when elongate members 120 are in the second geometry of FIG. 2. In a preferred embodiment, elongate members 120 are configured to, when transitioning from the first geometry of FIG. 1 to the second geometry of FIG. 2, cut through cancellous bone but avoid cutting through cortical bone. The mid portion of elongate members 120 defines a circular cross section in the second geometry shown in FIG. 2. In an alternative embodiment, the mid portion of elongate members 120 defines a non-circular cross section in the second geometry, such as an oval or rectangular cross section. These non-circular cross sections can correlate to similar non-circular cross sections when elongate members exist in the geometry of FIG. 1, or the corresponding cross sections can be dissimilar, such as a rectangular cross section in the first geometry and a circular cross section in the second geometry. The elongate members 120 of FIGS. 1 and 2 have a relatively flat, rectangular cross section that is similar to all the elongate members 120. In an alternative embodiment, elongate members 120 may have non-circular cross sections such as rectangular, triangular or oval cross sections. In another alternative embodiment, the cross section of a first elongate member 120 differs than the cross section of a second elongate member. Elongate members 120 may have similar or dissimilar widths, thicknesses, diameters, major and minor axis lengths, and materials of construction.

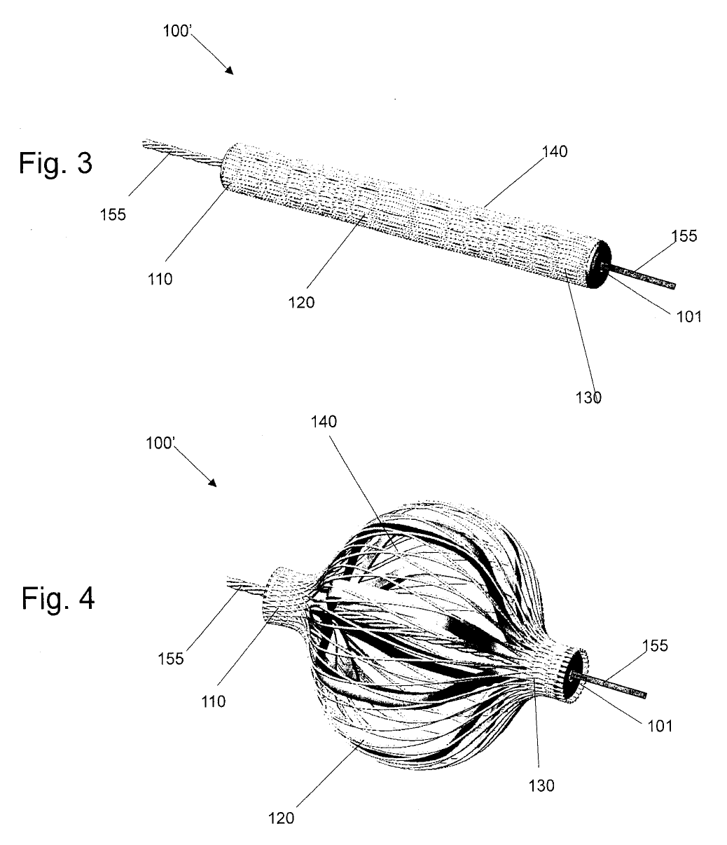

Referring now to FIG. 3, another preferred embodiment of the present invention is illustrated. Expandable cage 100′ is of similar construction to expandable cage 100 of FIG. 1 with the addition of a surrounding mesh, covering 140. Expandable cage 100′ is shown residing over guidewire 155, such as a K-wire used to access a vertebral segment in a percutaneous spine procedure. Guidewire 155 has been inserted through lumen 101 of expandable cage 100′. Covering 140, preferably a durable, foldable or expandable covering such as a metallic or non-metallic covering. Applicable materials for covering 140 include but are not limited to: metal such as a superelastic alloy; woven polyester such as Dacron; graphite and other carbon fibers; para-aramid fibers such as Kevlar; fluorocarbon fibers; polyethylene fibers such as ultra high molecular weight polyethylene fibers such as Dyneema produced by Royal DSM of The Netherlands; heat fused fibers; synthetic fibers; and combinations thereof and the other components of expandable cage 100′, are configured to be inserted into the interior body region of a patient in a first geometry as shown in FIG. 3.

Referring now to FIG. 4, the mid portion of elongate members 120 and covering 140 are configured to also transition to a radially expanded state depicted in FIG. 4. Covering 140 may include sharpened or abrasive edges such as to aid in cutting through bone or other body material. Covering 140 may be attached to a source of power, now shown, such as radiofrequency energy delivered in a monopolar and/or bipolar mode to aid in cutting through bone. In an alternative embodiment, covering 140 is resiliently biased in the compact first geometry of FIG. 3 or in the radially expanded geometry of FIG. 4.

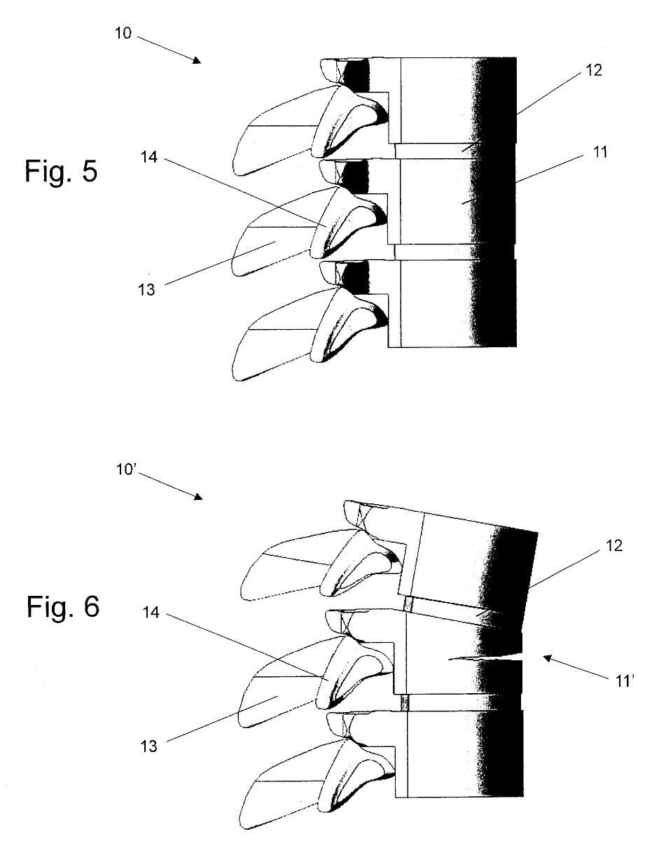

Referring now to FIG. 5, a spine portion of a healthy patient including 3 spinal motion segments is illustrated. Each spinal motion segment includes transverse processes 14, spinous process 13, intervertebral disc 12 and vertebral segment 11. Referring now to FIG. 6, the spine portion of FIG. 5 is illustrated in which a compromised middle vertebra, fractured vertebra 11′ is included in spine portion 10′. The interior or cancellous bone portion of fractured vertebra 11′ is a typical interior body region for insertion of the fixation apparatus of the present invention. Other applicable interior body regions include the internal portions of arm and leg bones, pelvis of a patient.

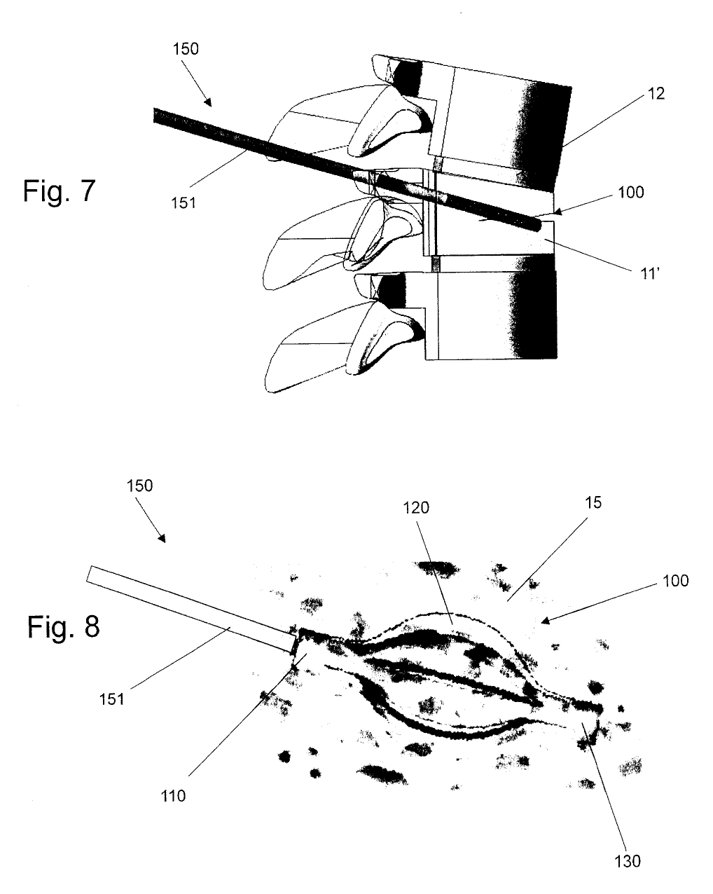

Referring now to FIG. 7, the fixation apparatus of the present invention, attached to a preferred delivery device, has been inserted into a compromised vertebra, such as over a K-wire, by way of a posterior approach and followed by sequential tubular dilators with final placement of working channel tube. Delivery device 150 includes a push rod, delivery tube 151, preferably a rigid elongate tube, with a distal end attached to expandable cage 100. Delivery device 150 may be inserted through a cannula, not shown but preferably a cannula approximately 4 mm in diameter or less. In an alternative embodiment, delivery tube 151 may be flexible along a majority of its length, or include hinged portions to allow flexing of delivery tube 151. As shown in FIG. 7, delivery tube 151 introduces expandable cage 100 along the axis of delivery tube 151, such that the axis of expandable cage 100 and the axis of the distal end of delivery tube 151 are aligned. In a preferred embodiment, the distal end of expandable cage 100 is configured to assist in delivery, such as a sharpened or other cutting surface on the distal end of distal ring 130 to aid in advancement of expandable cage 100 through bone. Delivery tube 151 is removably attached to expandable cage 100 such as via threaded engagement and/or frictional engagement. Delivery tube 151 is rotationally engaged, can be disengaged, and preferably is configured to be re-engaged such as to remove the expandable cage 100 as part of a normal, successful temporary implant procedure or in the case of a failed delivery. In an alternative embodiment, the distal end of delivery tube 151 is frictionally engaged with expandable cage 100, such that a retraction force is used to disengage delivery tube 151 from expandable cage 100.

Referring now to FIG. 8, the mid portions of elongate members 120 of expandable cage 100 have been partially expanded to define a greater radius than the first geometry shown in FIG. 7, such as by the application of an axial, pushing force on the proximal end of delivery device 150, proximal end not shown. In an alternative embodiment, delivery tube 151 is removably attached to distal ring 130, and an axial pulling force applied to the proximal end of delivery device 150 causes the partial expansion of the elongate members 120. In another alternative embodiment, delivery device 150 is attached to both proximal ring 110 and distal ring 130 and the application of both a pulling axial force and a pushing axial force is used to expand the mid portions of the elongate members 120. In another alternative embodiment, a separate device, not shown but configured to operably apply a radially outward force upon the elongate members 120, is inserted within the lumen defined by the elongate members 120. Such expansion devices include but are not limited to: an expandable balloon device, an expandable cage, and insertable bone reinforcing material. The insertable bone reinforcing material may be configured to expand upon insertion, such as a flowable material that is mixed at the insertion site and results in a chemical reaction including a volume increase and hardening. These expansion devices are preferably configured to be inserted within a lumen of delivery tube 151, and removed after sufficient expansion of elongate members 120.

As shown in FIG. 8, the elongate members 120 are cutting cut through the cancellous bone 15 of the vertebra, expanding radially out from the axis of the distal end of delivery tube 151. The sharpness and other cutting properties of elongate members 120 is configured to traverse through the cancellous bone, and preferably configured to avoid cutting through cortical bone. In an alternative embodiment, the elongate members 120 may radially expand at an axis different than the axis of delivery tube 151. In a preferred embodiment, the elongate members are sized and treated (such as a sharpening treatment) such as to cut through the cancellous bone without creating a void within the bone. In this preferred embodiment, the expandable cage 100 may be configured to provide structural reinforcement of the compromised vertebra without additional implanted devices or materials. Alternatively, additional materials such as a bone penetrating epoxy or cement may be added to further reinforce the vertebra. In an alternative embodiment, the elongate members are sized and treated to create a void in the interior body region, the void being filled with a subsequent device or material to further provide reinforcement and fixation.

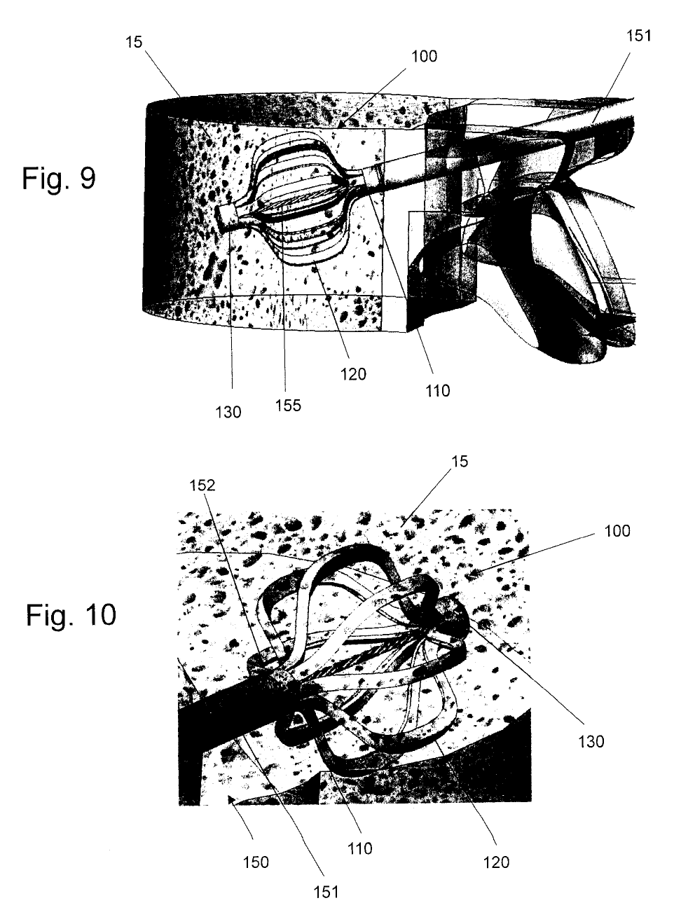

Referring now to FIGS. 9 and 10, a subsequent step to that shown in FIG. 8 is illustrated in which the expandable cage 100 has been fully expanded such that the mid portion of the elongate members 120 are in a second geometry, having cut through cancellous bone 15 without creating a void. FIG. 10 is a magnified view of the expandable cage 100 of FIG. 9. Attached to expandable cage 100 is delivery tube 151, which has a distal end configured to disengage from expandable cage 100, such as after the elongate members transition from the first geometry to the second geometry. Elongate members 120 are configured to provide structural reinforcement to the compromised vertebra, and in a preferred embodiment, delivery tube 151 is removed and the fixation procedure is complete. Expandable cage 100 may be configured to be accessed at a later date, such as in a subsequent procedure less than 30 days from the original implant, or at a date greater than 30 days from the original implant. These subsequent procedures may involve accessing expandable cage 100 to change the expanded geometry and/or introduce additional devices or materials. The subsequent procedure may include the removal of expandable cage 100, such as after the elongate members are transitioned from the expanded second geometry (of FIG. 2) to the compact first geometry (of FIG. 1).

In an alternative embodiment, implantation of additional fixation apparatus is performed to supplement the reinforcing function of expandable cage 100. In another alternative embodiment, elongate members 120 are configured to create a void or partial void in the cancellous bone, such as a void subsequently filled with a bone reinforcing agent. In yet another alternative embodiment, expandable cage 100 provides a temporary fixation function and is removed after the additional fixation apparatus are implanted. Delivery tube 151 is preferably configured to re-engage expandable cage 100, such as a re-engagement assisted by delivery of delivery tube once again over guidewire 155. Delivery tube 151 is configured to apply a force to cause elongate members 120 to transition from the second geometry (as shown in FIG. 2) back to the first geometry (as shown in FIG. 1), such that expandable cage 100 can be in a compact state and withdrawn from the patient. In an alternative embodiment, a second expandable cage 100 may be implanted within the same vertebra, such as to provide enhanced fixation.

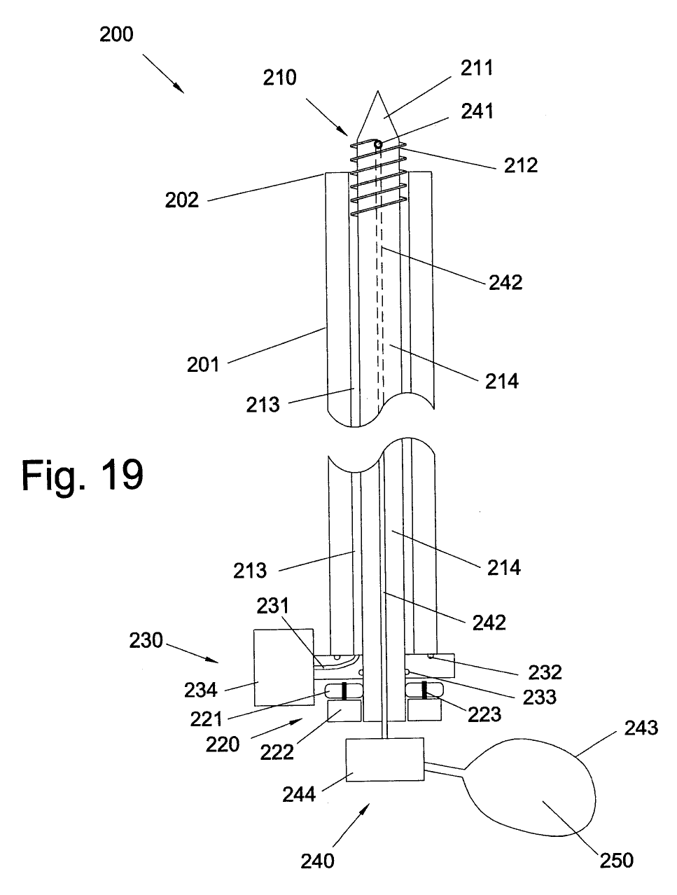

Referring now to FIG. 11, an alternative embodiment of the fixation apparatus of the present invention is illustrated, including the expandable cage 100′ of FIGS. 3 and 4, wherein the elongate members are surrounded by a mesh, covering 140. Covering 140 and elongate members 120 are configured to radially expand outward from the central axis of expandable cage 100, such as an expansion that causes covering 140 and elongate members 120 to cut through the cancellous bone of fractured vertebra 11′ without creating a void. As shown in FIG. 11, expandable cage 100 has been inserted into and expanded within a compromised vertebra, fractured vertebra 11′, in a similar method as described in reference to FIGS. 7 through 10. The percutaneous delivery device has been removed, and a dispenser device has been inserted. The dispenser device, such as the dispenser device 200 of FIG. 19, may be configured to deliver a substance into bone without creating a void in the bone. The dispenser device includes shaft 201 with distal end 202 engaged with the proximal ring 110 of expandable cage 100. In a preferred embodiment, shaft 201 is introduced over a guidewire, such as the same K-wire used to introduce the delivery device of FIG. 7. In an alternative embodiment, delivery device 150 of FIGS. 7 through 10 is used to deliver reinforcing material, obviating the need for a second inserted device. Shaft 201 may be rigid or flexible along a majority of its length, or it may include flexible or hinged portions. Dispenser device shaft 201 distal end 202 is preferably rotationally or frictionally engaged with proximal ring 110. In an alternative embodiment, distal end 202 simply passes through proximal ring 110 and is advanced to a location proximate distal ring 130. Reinforcing material 250, preferably a bone penetrating material configured to provide structural integrity to cancellous bone, is being introduced within the bone portion surrounded by expandable members 120. Reinforcing material 250 may or may not harden over time. Reinforcing material 250 may be a flowable material or may be a material requiring advancement through shaft 201 with a push rod device, not shown. Reinforcing material 250 may be inserted into a void region and/or it may be configured to penetrate bone such as cancellous bone. Reinforcing material 250 may comprise a plurality of rigid, solid particles, such as solid particles deliverable through a tube less than 4 mm. Reinforcing material 250 may comprise a bone material and/or a bone-hardening agent. Reinforcing material 250 may comprise a first part and a second part, such as a two-part material configured to change properties once mixed.

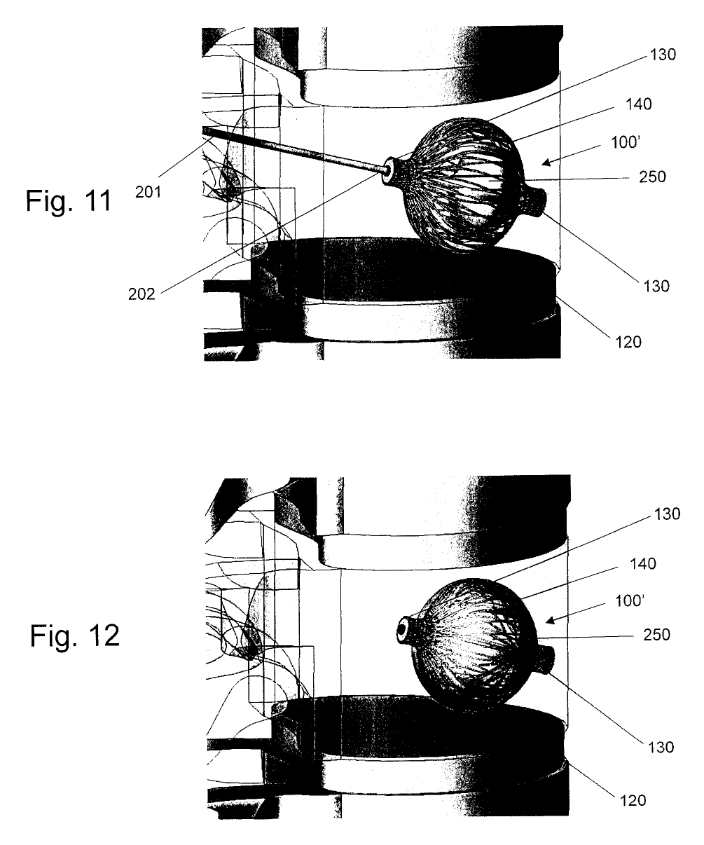

Referring now to FIG. 12, the final amount of reinforcing material 250 has been inserted within the interior region of elongate members 120. The dispenser device and shaft 201 has been disengaged from expandable cage 100 and removed from the patient. The original insertion guidewire and any percutaneous cannula have been removed completing the procedure. Reinforcing material 250 may be in its final state, or may change properties such as rigidity over time. Reinforcing material 250 may be biodegradable, or may include a biodegradable portion. Reinforcing material 250 may be relatively homogenous, or may include portions with different composition and/or properties. Reinforcing material 250, when injected or at a subsequent time, may apply a radial outward force which further expands the elongate members 120. In an alternative embodiment, a second expandable cage 100, with or without reinforcing material 250, may be implanted in the same vertebra to provide additional fixation.

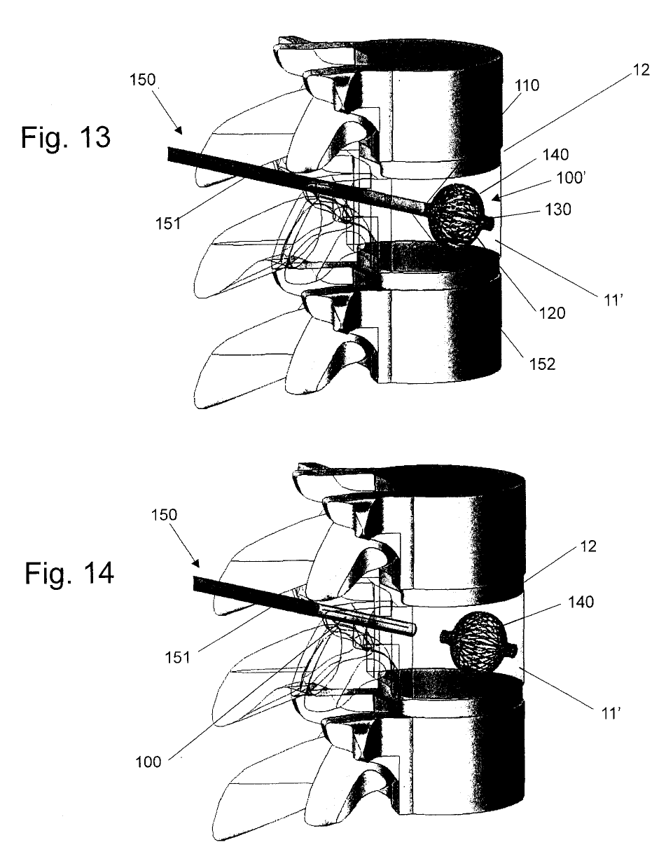

Referring now to FIGS. 13 through 16, an alternative method of the present invention is illustrated in which the expandable cage 100 is removed leaving covering 140 in place within the fractured vertebra 11′. In FIG. 13, expandable cage 100 has been inserted into cancellous bone of fractured vertebra 11′ and fully expanded such as has been described in reference to FIGS. 7 through 10. Expandable cage 100 remains attached to distal end 152 of delivery tube 151 of delivery device 150. Distal end 152 is engaged with proximal ring 110, such as via a threaded, rotational engagement, a frictional engagement, or other engagement means. Distal end 152 can be operably disengaged from proximal ring 110, and preferably can be re-engaged. In an alternative embodiment, distal end 152 is operably engaged with distal ring 130, or another portion of expandable cage 100. Delivery device 150 is configured to cause the elongate members 120 of expandable cage 100 transition from a first geometry to a second geometry with the application of an axial pushing force. In the embodiment of FIGS. 13 through 16, delivery device 150 is further configured to cause the elongate members 120 of expandable cage 100 to also transition from the second geometry back to the first geometry. Referring now to FIG. 14, the expandable cage has been transitioned back to the first geometry, and is being withdrawn from the patient after having been removed from within covering 140. This withdrawal step may be performed within twenty-four hours, within thirty days, or at a time greater than thirty days past the original implantation procedure. Covering 140 is configured to provide a structural reinforcement to an internal portion of fractured vertebra 11′. In a preferred embodiment, a guidewire, not shown, remains with its distal portion within covering 140.

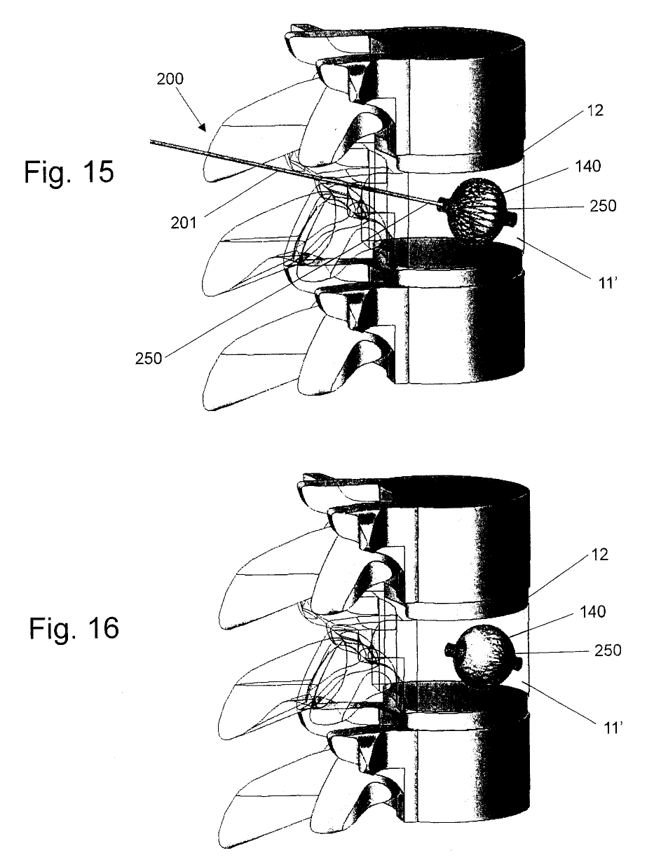

Referring now to FIG. 15, dispenser device 200 has been percutaneously inserted into fractured vertebra 11′ and the distal end of shaft 201 resides within expanded covering 140, which is configured to receive one or more types of reinforcing material. The dispenser device, such as the dispenser device 200 of FIG. 19, may be configured to deliver a substance into bone without creating a void in the bone. Reinforcing material 250, such as a bone penetrating or void filling agent, is being introduced within the space defined by expanded covering 140. Referring now to FIG. 16, the final volume of reinforcing material 250 has been injected, and dispenser device 200 has been removed. The fixation procedure is complete, although the reinforcing material 250 properties may change over time, as has been described hereabove. Covering 140 and/or reinforcing material 250 may be biodegradable or include biodegradable portions. Covering 140 and/or reinforcing material 250 may remain implanted for a subchronic period less than 30 days, or remain as a chronic implant. In an alternative embodiment, a second covering 140 may be implanted within the same fractured vertebra 11′.

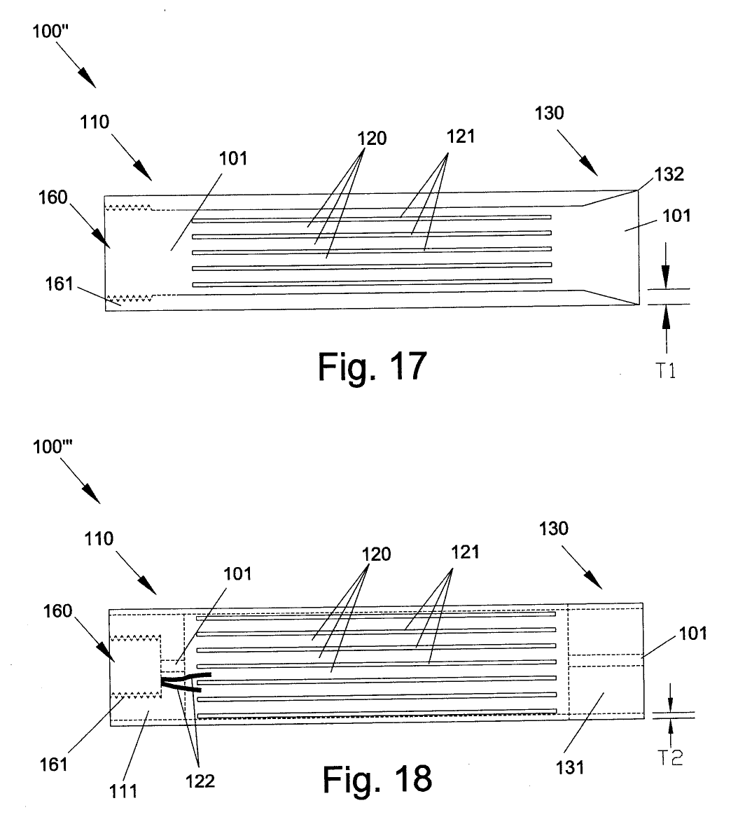

Referring now to FIG. 17, a preferred embodiment of the fixation apparatus of the present invention is illustrated. Expandable cage 100″, shown in a first geometry, is preferably manufactured from a metal tube such as a Nitinol tube with a thickness TI of approximately 0.05 mm to 2 mm on the proximal end of expandable cage is proximal ring 110 that includes engagement portion 160 comprising a set of internal threads 161. Threads 161 are configured to operably engage the distal end of a percutaneously inserted threaded tube, such as the distal end of the delivery device and/or dispensing device as described in reference to the previous figures. Alternatively or additionally, other attachment means are employed, in the proximal ring 110, the distal ring 130, or at a location between proximal ring 110 and distal ring 130. Slots 121 have been made in the tube, creating multiple elongate members 120 between the slots 121. The multiple elongate members 120 have proximal ends which attach to the proximal ring 110, and distal ends which attach to distal ring 130. The elongate members 120 are configured to be transitioned from the first geometry (shown) to a second geometry (as depicted in FIG. 2) by applying an axial force to either the proximal ring 110, distal ring 130, or both, or by providing a radially outward force from within the lumen defined by the elongate members 120. The elongate members may have similar or dissimilar properties such as widths and thicknesses. The elongate members may be sharpened or otherwise treated such as to facilitate cutting through bone. The elongate members are shown in a first geometry, a compact state for introduction through a cannula or other tube, and can be radially expanded to a second geometry, such as via a natural bias, or through a transformation such as a plastic deformation.

Distal ring 130 has a sharpened edge 132, which is used to assist in advancement through bone or other tissue, such as advancement over a guidewire passing through lumen 101. Sharpened edge 132 may include serrations, abrasive material such as diamond chips, or other enhancements to improve cutting through bone. The tube used to manufacture expandable cage 100″ may be extruded, molded, assembled from a flat sheet that is rolled and welded along a seem, made by creating a hole in a solid rod such as with machining, drilling such as laser drilling, etching, or constructed in a layering process such as stereolithography. Slots 121 may be made in a machining process, a laser cutting process, a sawing operation or an etching process. The tube may be resiliently biased, such as in the first geometry, the second geometry, or a geometry in between, such as a heating process used to “train” Nitinol into a specific resiliently biased shape. The elongate members 120 may have similar or dissimilar properties, such as widths, thickness and other cross sectional profile properties. The manufacturing process of expandable cage 100″ may further include a sharpening process used to sharpen the elongate members to assist in their radial expansion through bone, such as a radial expansion that does not create a void in bone. The manufacturing process may include attachment of an electrical connection for attaching one or more elongate members 120 to a supply of energy to assist in deployment through bone. The manufacturing process may include the creation of an attachment element, such as threads 161 on proximal ring 110, or an attachment element at another location such as on or proximate to distal ring 130. Attachment elements may consist of: threaded elements; bayonet locks; frictional engagement tubes; fusible links; and combinations thereof.

Further manufacturing processed may be employed, such as a material treatment process, such as annealing, used to modify the stiffness of the entirety or a portion of expandable cage 100″. The external surface of expandable cage 100″ may be modified, such as a treatment that removes material such as etching, or a treatment that adds material such as plating or anodizing. Material can be added or removed to a portion of expandable cage 100″, such as a material deposition process that adds abrasive material, only to the elongate members 120. A material shape forming process may be used such as a process that places the elongate members 120 in a circular, oval or rectangular cross section. A mesh or other covering, not shown, may be added to the entirety or a portion of expandable cage 100″. The mesh may be flexible, or may be sized to allow sufficient radial expansion of the elongate members 120. The mesh may be selected from one or more biocompatible materials, such as materials selected from the group consisting of: metal; Dacron; graphite and other carbon fibers; para-aramid fibers such as Kevlar; fluorocarbon fibers; polyethylene fibers such as ultra high molecular weight polyethylene fibers such as Dyneema produced by Royal DSM of The Netherlands; heat fused fibers; synthetic fibers; and combinations thereof. The mesh may be configured to be electrified and/or may include an abrasive coating such as diamond particles to assist in cutting through bone.

Referring now to FIG. 18, another preferred embodiment of the fixation apparatus of the present invention is illustrated. Expandable cage 100′″ is preferably manufactured from a metal tube such as a Nitinol tube with a thickness T2 that is preferably less than the thickness T1 of Expandable cage 100″ of FIG. 17. On the proximal end of expandable cage, proximal plug 111 has been inserted, such as via a swaging or welding process, to create proximal ring 110. Included in proximal plug 11 is engagement portion 160 comprising a set of internal threads 161. Threads 161 are configured to operably engage the distal end of a percutaneously inserted threaded tube, such as the distal end of the delivery device and/or dispensing device as described in reference to the previous figures. Alternatively or additionally, other attachment means are employed, in the proximal ring 110, the distal ring 130, or at a location between proximal ring 110 and distal ring 130. Also included in proximal plug 111′ are wires 122, connected to one or more elongate members 120, and configured to attach to a supply of electrical energy provided by a delivery device, not shown. The energy, preferably monopolar and/or bipolar radiofrequency energy, is used to assist in radial expansion of the elongate members 120 from the first geometry (shown) to the second geometry (as depicted in FIG. 2).

Slots 121 have been made in the tube, creating the multiple elongate members 120 between the slots 121. The multiple elongate members 120 have proximal ends that attach to the proximal ring 110, and distal ends which attach to distal ring 130. The elongate members 120 are configured to be transitioned from the first geometry to a second geometry by applying an axial force to either the proximal ring 110, distal ring 130, or both, or by providing a radially outward force from within the lumen defined by the elongate members 120. The elongate members may have similar or dissimilar properties such as widths and thicknesses. The elongate members may be sharpened or otherwise treated such as to facilitate cutting through bone. The elongate members are shown in a first geometry, a compact state for introduction through a cannula or other tube, and can be radially expanded to a second geometry, such as via a natural bias, or through a transformation such as a plastic deformation.

Distal ring 130 includes distal plug 131 which has been inserted utilizing a similar process used to insert proximal plug 111 in proximal ring 110. Both proximal plug 111 and distal plug 131 include a guidewire lumen, lumen 101, used to insert expandable cage 100′″ over a guidewire such as a K-wire used in spinal access procedures. The various enhancements such as material treatments, additions such as a surrounding mesh, and other manufacturing processes described in reference to expandable cage 100″ of FIG. 17, may be employed, in whole or in part, in the manufacturing of expandable cage 100′″ of FIG. 18.

Referring now to FIG. 19, a preferred embodiment of a device for inserting material into bone is illustrated. Dispenser device 200 includes an elongate shaft 201 with distal end 202. Shaft 201, preferably a rigid tube, but alternatively a flexible tube or a tube with one or more flexible hinged portions, slidingly receives auger assembly 210. Auger assembly 210 has at its distal end, tip 211, preferably a sharpened and/or abrasively (e.g. diamond) coated tip configured to drill into bone. Proximate tip 211 are cutting threads 212, configured to, when rotated as sufficient angular velocity, pulverize bone into small particles and direct those particles into space 213, which is located between shaft 214 of auger assembly 210 and shaft 201. The proximal end of auger assembly 210 is attached to a rotational drive assembly 220 consisting of one or more rotational motors 222, each of which is operably attached to an axle 223 which in turn is operably attached to a drive wheel 221. Drive wheel 221 is frictionally engaged with shaft 214 such that rotation of the drive wheel 221 causes rotation of shaft 214, as well as cutting threads 212 and distal end 211. Other forms of rotating auger assembly 210 can be employed without departing from the spirit and scope of the embodiment.

Dispenser device 200 includes a reinforcing material delivery function including material delivery assembly 240 configured to deliver one or more agents to an interior body region of a patient. Material delivery assembly 240 is in fluid communication with reservoir 243 that includes reinforcing material 250. Reinforcing material 250 consists of one or more materials such as: glues and epoxies such as 2-part epoxies; cements such as bone cements; bone biologic material; artificial bone material; bone penetrating agents; solid particle based fillers; and curable materials. Housing 244 includes material injection means such as a pump, auger drive, or other material propulsion means configured to propel material from reservoir 243 and into lumen 242 of shaft 214. The flowable material will travel distally through lumen 242 and exit through one or more exit holes 241 located within threads 212.

Dispenser device 200 includes a bone material evacuation function including vacuum assembly 230. Vacuum assembly 230 includes housing 234 which surrounds vacuum means in fluid communication with lumen 231 which is further in fluid communication with space 213 located between shaft 214 and shaft 201. Fluid seals 232 and 233 are provided to maintain the vacuum provided by vacuum assembly 230 as well as allow rotation of shaft 201 and shaft 214. Seals 232 and 233 are preferably a compressible O-ring, such as an elastomeric O-ring, which are received in grooves of vacuum assembly 230 and are sized to create a sufficient fluid seal. The vacuum provided by vacuum assembly 230 causes the material directed into space 213 by threads 212 to move proximally and be captured within housing 234 of vacuum assembly 230. Controls, not shown, are included to allow an operator to activate vacuum assembly 230, material delivery assembly 240, and rotational drive assembly 220. In a preferred embodiment, a single control is included, also not shown, that causes the vacuum assembly, material delivery assembly 240 and the rotational drive assembly 220 to be activated simultaneously. In this preferred embodiment, auger assembly 210 is rotated while tip 211 is advanced into bone; bone material is pulverized by threads 212 and this pulverized material directed into space 213 and vacuum driven into housing 234; reinforcing material 250 is advanced down lumen 242 and out of exit hole 241; all simultaneously. In this preferred configuration; reinforcing material can be introduced to an interior body region of a patient without ever creating a void space. In another preferred embodiment, an interlock is provided that prevents the auger assembly 120 rotation and/or the vacuum assembly 230 activation to occur without material delivery assembly 240 being on and reinforcing material being delivered to the interior body region via exit hole 241. In another preferred embodiment, activation of a control causes the material delivery assembly 240 to be activated prior to vacuum assembly 230 and/or auger assembly 210 being activated, assuring that agent is injected prior to any body material being removed.

Auger assembly 210 can be advanced or retracted by an operator, such as by advancing or retracting drive assembly 220, with a fluid seal maintained between shaft 214 and vacuum assembly 230 via gasket 233. Advancement of auger assembly 210 causes tip 211 to extend beyond distal end 202 of shaft 201, and retraction causes tip 211 to reside within shaft 201. The auger assembly 210 of FIG. 19 is shown in the fully advanced position. In a preferred method, sequential advancement of dispenser device 200 and subsequent advancement of only auger assembly 210 are made to dispense material into an interior body region of a patient. In an alternative embodiment, shaft 201 and/or shaft 214 of auger assembly 210 includes deflection means, such as a pull-wire deflection assembly, to provide angular orientation control to an operator, in addition to the ability to advance and retract dispenser device 200 and auger assembly 210. Angular orientation may simplify dispensing of material into a three dimensional space such as a sphere.

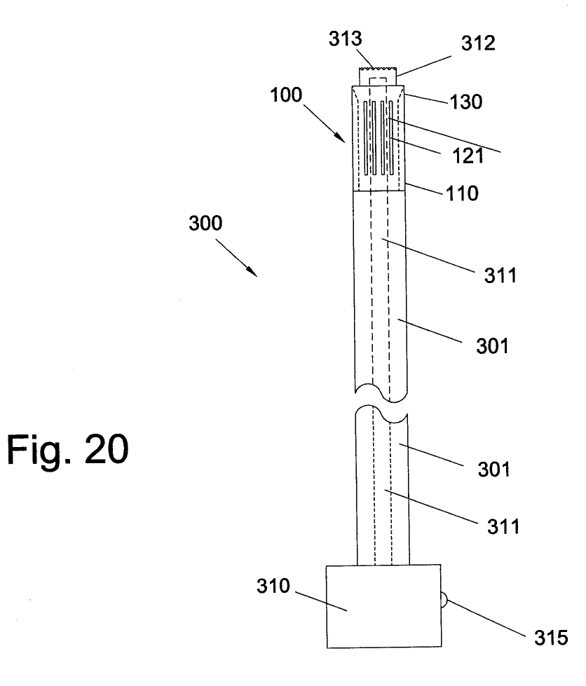

Referring now to FIG. 20, a fixation apparatus delivery device is illustrated. Delivery device 300 includes shaft 301, preferably a rigid shaft sized for introduction to an interior body region of a patient through a cannula such as a 4 mm cannula. In an alternative embodiment, shaft 301 may be flexible along a majority of its length, or may include one or more flexible or hinged portions for introduction into an interior body space requiring flexion of shaft 301. Attached to the distal end of shaft 301 is an expandable cage 100 of the present invention shown in a first geometry, including slots 121, radially expandable elongate members 120, proximal ring 110 and distal ring 130. The distal end of shaft 301 is removably attached to proximal ring, such as via threaded engagement or other engagement means described in detail in reference to various embodiments hereabove. Distal ring 130 may include one or more enhancements on its distal, leading edge configured to assist in deploying expandable cage 100 into bone. Such enhancements include but are not limited to: sharpened edge, serrated edge, abrasive material such as diamond chips adhered to edge, and other improvements.

On the proximal end of delivery device 300 is drive assembly 310, which is operably attached to internal drive shaft 311. The distal end of drive shaft 311 includes cutting element 312, which includes serrations 313. Cutting element 312 extends beyond the distal end of distal ring 130, such that serrations 313 make contact with bone as delivery device 300 is advanced into bone. Rotation of cutting element 312 via rotation of shaft 301 by drive assembly 310 is activated by an operator depressing switch 315. Advancement of delivery device 300 into bone while cutting element 312 is rotating decreased the required axial force for insertion of expandable cage 100 into bone, such as decreased force that prevents premature radial expansion of elongate members 120. Once expandable cage 100 is in place, cutting element 312 can have its rotation ceased, and potentially shaft 311 and cutting assembly 312 removed. Subsequent axial forward force, placed on shaft 301 and subsequently expandable cage 100 causes elongate members 120 to radially expand such that the mid portions of elongate members 120 form a larger diameter consistent with the second geometry described in reference to FIG. 2 of this application. In a preferred embodiment, cutting element 312, shaft 311 and drive assembly 310 include a thru lumen, not shown, configured to allow placement of delivery device 300 and expandable cage 100 over a guidewire, such as a K-wire introduced into the cancellous bone of a vertebra of a patient.

It should be understood that numerous other configurations of the devices and methods described herein may be employed without departing from the spirit or scope of this application. The devices and methods of the present invention are configured to be inserted without resection of tissue, however procedures including or requiring resection are also supported.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An apparatus for providing fixation of an interior body region of a patient, said apparatus comprising:
an expandable cage comprising:
a proximal ring;
a distal ring; and
a plurality of elongate members, each elongate member including a proximal end, a distal end and a middle portion therebetween, wherein each proximal end is fixedly attached to the proximal ring of the expandable cage and each distal end is fixedly attached to the distal ring of the expandable cage;
wherein the plurality of elongate members are transitionable from a first geometry with the plurality of the middle portions defining a first area to a second geometry with the plurality of middle portions defining a second area; and
wherein the second area is larger than the first area.

2. The apparatus of claim 1 wherein the first area or the second area approximate a circle.

3. The apparatus of claim 1 wherein the first area or the second area approximate a rectangle.

4. The apparatus of claim 1 wherein the first area or the second area approximate an oval.

5. The apparatus of claim 1 wherein the expandable cage is configured to provide structural reinforcement of an interior body region when in the second geometry.

6.-132. (canceled)

133. A method of manufacturing an expandable cage for placement into an interior body region of a patient comprising:
creating a hollow tube with a proximal end and a distal end; and
creating multiple slots that extend from a point distal said tube proximal end and proximal to said tube distal end, said slots creating a plurality of elongate members each with a middle portion;
wherein the plurality of middle portions are expandable from a first geometry defining a first area to a second geometry defining a second area greater than the first area.

134. The method of claim 133 wherein the first area or the second area approximates a circle.

135. The method of claim 133 wherein the first area or the second area approximates a rectangle.

136. The method of claim 133 wherein the first area or the second area approximates a oval.

137. The method of claim 133 wherein the tube is created with an extrusion operation.

138.-174. (canceled)

175. A method of providing fixation to an interior body region of a patient, said method comprising:
providing the apparatus of claim 1;
inserting the apparatus into the interior body region of the patient; and
causing the elongate members of the expandable cage to transition from the first geometry to the second geometry.

176. The method of claim 175 wherein the interior body region is a fractured or impending fracture bone segment.

177. The method of claim 175 wherein the interior body region is a segment of osteoporotic bone.

178. The method of claim 175 wherein the interior body region is a portion of a bone in a limb of the patient.

179. The method of claim 175 wherein the interior body region is a cancellous bone portion of a vertebra of the patient.

180.-189. (canceled)

190. A device for placing material into an interior body region of a patient comprising:
an elongate tube with a proximal end and a distal end;
a body material pulverizing assembly deployable from the distal end of the elongate tube and configured to contact and pulverize material proximate said distal end;
a vacuum assembly configured to remove the pulverized body material from the interior body region; and
an agent delivery assembly configured to deliver an agent to the interior body region in the space previously occupied by the pulverized material;
wherein the agent is placed into the interior body region prior to a void being created in said interior body region.

191. The device of claim 190 wherein the elongate tube is rigid along a majority of its length.

192. The device of claim 190 wherein the elongate tube is flexible along a majority of its length.

193. The device of claim 190 wherein the elongate tube includes at least one hinged portion along its length.

194. The device of claim 190 wherein the elongate tube distal end is configured to be deflected by an operator.

195.-212. (canceled)
